(19) United States
(12) Patent Application Publication
(10) Pub. No.: US 2009/0186075 A1
(43) Pub. Date: Jul. 23, 2009

(54) Composition

(76) Inventor: Ivan M. Roitt, London (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/086,736

(22) PCT Filed: Dec. 21, 2006
(86) PCT No.: PCT/GB2006/004861

§ 371 Date: Jan. 14, 2009

(30) Foreign Application Priority Data
Dec. 22, 2005 (GB) 0526188.8
Dec. 23, 2005 (GB) 0526395.9

(51) Int. Cl. A61K 9/127; A61K 38/17; A61K 39/395; C07K 14/725; C07K 16/00

(52) U.S. Cl. 424/450; 514/12; 424/130.1; 530/402; 530/390.5; 424/133.1; 424/158.1

(57) ABSTRACT

The present invention provides the use of a composition comprising an antigen recognizing molecule or fragment thereof and a lipid-based carrier for blocking an interaction between a ligand and a receptor.

The present invention relates to the field of compositions and methods for blocking interactions between a ligand and a receptor and for the optimal targeting of cell surface molecules. In particular, the present invention relates to therapeutic agents comprising immunoglobulins or T-cell receptors or fragments thereof and methods of their administration.

There has been considerable interest in recent years in the use of monoclonal antibodies for therapeutic applications. Conventional antibodies are large multi-subunit protein molecules comprising at least four polypeptide chains. For example, human IgG has two heavy chains and two light chains that are disulfide bonded to form the functional antibody. The size of a conventional IgG is about 150 kD. Because of their relatively large size, complete antibodies (e.g. IgG, IgA, IgM,) are limited in their therapeutic usefulness due to problems in, for example, tissue penetration. Considerable efforts have focused on identifying and producing smaller antibody fragments that retain antigen binding function and solubility.

The heavy and light polypeptide chains of antibodies comprise variable (V) regions that directly participate in antigen interactions, and constant (C) regions that provide structural support and function in non-antigen-specific interactions with immune effectors. The antigen binding domain of a conventional antibody is comprised of two separate domains: a heavy chain variable domain (V_{H}) and a light chain variable domain (V_{L}: which can be either Vκ or Vλ). The antigen binding site itself is formed by six polypeptide loops: three from the V_{H} domain (H1, H2 and H3) and three from the V_{L} domain (L1, L2 and L3). In vivo, a diverse primary repertoire of V genes that encode the V_{H} and V_{L} domains is produced by the combinatorial rearrangement of gene segments. C regions include the light chain C regions (referred to as C_{L} regions) and the heavy chain C regions (referred to as C_{H}1, C_{H}2 and C_{H}3 regions).

A number of smaller antigen binding fragments of naturally occurring antibodies have been identified following protease digestion. These include, for example, the “Fab fragment” (V_{L}˜C_{L}˜C_{H}1˜V_{H}), “Fab′ fragment” (a Fab with the heavy chain hinge region) and “F(ab′)₂ fragment” (a dimer of Fab′ fragments joined by the heavy chain hinge region). Recombinant methods have been used to generate even smaller antigen-binding fragments, referred to as “single chain Fv” (variable fragment) or “scFv”, consisting of V_{L} and V_{H} joined by a synthetic peptide linker.

While the antigen binding unit of a naturally-occurring antibody (e.g., in humans and most other mammals) is generally known to be comprised of a pair of V regions (V_{L}/V_{H}), camelid species express a large proportion of fully functional, highly specific antibodies that are devoid of light chain sequences. The camelid heavy chains antibodies are found as homodimers of a single heavy chain, dimerized via their constant regions.

The variable domains of these camelid heavy chain antibodies are referred to as V_{HH} domains and retain the ability, when isolated as fragments of the V_{H} chain, to bind antigen with high specificity (Hamers-Casterman et al., 1993, Nature 363: 446-448; Gahroudi et al., 1997, FEBS Lett. 414: 521-526). Antigen binding single V_{H} domains have also been identified from, for example, a library of murine V_{H} genes amplified from genomic DNA from the spleens of immunized mice and expressed in E. coli (Ward et al., 1989, Nature 341:544-546). Ward et al. named the isolated single V_{H} domains “dAbs,” for “domain antibodies”. The term “dAb” will refer herein to a single immunoglobulin variable domain (V_{H} or V_{L}) polypeptide that specifically binds antigen. A “dAb” binds antigen independently of other V domains; however, as the term is used herein, a “dAb” can be present in a homo- or heteromultimer with other V_{H} or V_{L} domains where the other domains are not required for antigen binding by the dAb, i.e., where the dAb binds antigen independently of the additional V_{H} or V_{L} domains.

Single immunoglobulin variable domains, for example, V_{HH}, are thus very small antigen-binding antibody units (minibodies just have 2 hypervariable regions). In principle, for use in therapy, human antibodies are preferred, primarily because they are not as likely to provoke an immune response when administered to a patient, but as they are selected from naïve libraries, a process of mutation and selection is normally required to improve the affinity. As noted above, isolated non-camelid V_{H} domains tend to be relatively insoluble and are often poorly expressed. Comparisons of camelid V_{HH} with the V_{H} domains of human antibodies reveals several key differences in the framework regions of the camelid V_{HH} domain corresponding to the V_{H}/V_{L} interface of the human V_{H} domains. Mutation of these residues of human V_{H}3 to more closely resemble the V_{HH} sequence (specifically Gly44 to Glu, Leu45 to Arg and Trp47 to Gly) has been performed to produce “camelized” human V_{H} domains that retain antigen binding activity (Davies & Riechmann, 1994, FEBS Lett. 339: 285-290) yet have improved expression and solubility. Variable domain amino acid numbering used herein is consistent with the Kabat numbering convention (Kabat et al., 1991, Sequences of Immunological Interest, 5th ed. U.S. Dept. Health & Human Services, Washington, D.C.). WO 03/035694 (Muyldermans) reports that the Trp103 to Arg mutation improves the solubility of non-camelid V_{H} domains. Davies & Riechmann (1995, Biotechnology N.Y. 13: 475-479) also report production of a phage-displayed repertoire of camelized human V_{H} domains and selection of clones that bind hapten with affinities in the range of 100-400 nM, but clones selected for binding to protein antigen had weaker affinities.

Camelid V_{HH} domains are naturally soluble and can be derived as high affinity clones without the need for mutations other than minor changes to increase their homology with human V_{H} to largely eliminate their already very low potential immunogenicity for human use. Camelid V_{HH} also have the specially prominent H3 antigen-binding loops which facilitate their penetration of “canyons” characteristic of several viral species.

WO 00/29004 (Plaskin et al.) and Reiter et al. (1999, J. Mol. Biol. 290:685-698) describe isolated V_{H} domains of mouse antibodies expressed in E. coli that are very stable and bind protein antigens with affinity in the nanomolar range. WO 90/05144 (Winter et al.) describes a mouse V_{H} domain antibody fragment that binds the experimental antigen lysozyme with a dissociation constant of 19 nM.

WO02/051870 (Entwistle et al.) describes human V_{H} single domain antibody fragments that bind experimental antigens, including a V_{H} domain that binds an scFv specific for a Brucella antigen with an affinity of 117 nM, and a V_{H} domain that binds an anti-FLAG IgG.

Tanha et al. (2001, J. Biol. Chem. 276:24774-24780) describe the selection of camelized human V_{H} domains that bind two monoclonal antibodies used as experimental antigens and have dissociation constants in the micromolar range.

U.S. Pat. No. 6,090,382 (Salfeld et al.) describe human antibodies that bind humanTNF-a with affinities of 10⁻⁸ M or less, have an off-rate (Ko) for dissociation of human TNF-α of 10³ sec⁻¹ or less and neutralize human TNF-α activity in a standard L929 cell assay.

Despite these advances, immunoglobulins and their fragments are still typically expensive to produce commercially and vulnerable to aggregation, proteolysis (for instance at low pH) and denaturation. Thus there is still a need for new formulations and methods for delivery of immunoglobulins which improve their efficiency (i.e. require less antibody to have an equivalent therapeutic effect), and which are more stable, particularly with reference to their relative insensitivity to temperature or the acidic proteolytic environment of the stomach, important for oral administration.

Immunomicelles or liposomes have been used to target the delivery of therapeutic agents to particular types of cell (see Torchilin V. P. et al. Proc. Natl. Acad. Sci. 2003, 100, 6039 and U.S. Pat. No. 6,214,388). However, the antibody in the liposome or micelle is not used to provide a therapeutic effect in itself, but merely to ensure that the liposome or micelle binds to a cell type into which it is desired to deliver the therapeutic agent loaded in the micelle.

Accordingly the present invention provides use of a composition comprising an antigen recognizing molecule or fragment thereof and a lipid-based carrier for blocking an interaction between a ligand and a receptor.

In one embodiment the present invention provides use of a composition comprising an T cell receptor or fragment thereof and a lipid-based carrier for blocking an interaction between a ligand and a receptor.

In a further embodiment the present invention provides use of a composition comprising an immunoglobulin or fragment thereof and a lipid-based carrier for blocking an interaction between a ligand and a receptor.

In a further aspect, the present invention provides use of a composition comprising an antigen recognizing molecule or fragment thereof and a lipid-based carrier for the preparation of a medicament for preventing or treating a disease, wherein the disease is mediated by binding of a ligand to a receptor, and the composition is capable of blocking binding of the ligand to the receptor.

In a further aspect, the present invention provides use of a composition comprising a T cell receptor or fragment thereof and a lipid-based carrier for the preparation of a medicament for preventing or treating a disease, wherein the disease is mediated by binding of a ligand to a receptor, and the composition is capable of blocking binding of the ligand to the receptor.

In a further aspect, the present invention provides use of a composition comprising an immunoglobulin or fragment thereof and a lipid-based carrier for the preparation of a medicament for preventing or treating a disease, wherein the disease is mediated by binding of a ligand to a receptor, and the composition is capable of blocking binding of the ligand to the receptor.

In a further aspect, the present invention provides a method for inhibiting an interaction between a ligand and a receptor, comprising contacting the ligand and/or receptor with a composition comprising an antigen recognizing molecule or fragment thereof and a lipid-based carrier, wherein the antigen recognizing molecule or fragment thereof binds to the ligand and/or receptor, thereby inhibiting the interaction between the ligand and the receptor.

In a further aspect, the present invention provides a method for inhibiting an interaction between a ligand and a receptor, comprising contacting the ligand and/or receptor with a composition comprising a T cell receptor or fragment thereof and a lipid-based carrier, wherein the T cell receptor or fragment thereof binds to the ligand and/or receptor, thereby inhibiting the interaction between the ligand and the receptor.

In a further aspect, the present invention provides a method for inhibiting an interaction between a ligand and a receptor, comprising contacting the ligand and/or receptor with a composition comprising an immunoglobulin or fragment thereof and a lipid-based carrier, wherein the immunoglobulin or fragment thereof binds to the ligand and/or receptor, thereby inhibiting the interaction between the ligand and the receptor.

In a further aspect, the present invention provides a method for preventing or treating a disease, comprising administering to a subject a therapeutically effective amount of a composition comprising an antigen recognizing molecule or fragment thereof and a lipid-based carrier, wherein the disease is mediated by binding of a ligand to a receptor, and the composition blocks binding of the ligand to the receptor.

In a further aspect, the present invention provides a method for preventing or treating a disease, comprising administering to a subject a therapeutically effective amount of a composition comprising a T cell receptor or fragment thereof and a lipid-based carrier, wherein the disease is mediated by binding of a ligand to a receptor, and the composition blocks binding of the ligand to the receptor.

In a further aspect, the present invention provides a method for preventing or treating a disease, comprising administering to a subject a therapeutically effective amount of a composition comprising an immunoglobulin or fragment thereof and a lipid-based carrier, wherein the disease is mediated by binding of a ligand to a receptor, and the composition blocks binding of the ligand to the receptor.

In a further aspect, the present invention provides use of a composition comprising a T cell receptor or fragment thereof and a lipid-based carrier for delivery of a therapeutic agent to a cell.

In a preferred aspect this composition has any of the functional capabilities for a composition comprising an antigen recognizing molecule and a lipid-based carrier described herein.

In a further aspect, the present invention provides a composition comprising an immunoglobulin or fragment thereof and a lipid-based carrier, wherein the composition is capable of inhibiting binding of a ligand to a receptor, binding of the ligand to the receptor being associated with induction or progression of a human or animal disease.

In a further aspect, the present invention provides a composition comprising a T cell receptor or fragment thereof, a lipid-based carrier and optionally a therapeutic agent.

In a further aspect, the present invention provides a composition as defined anywhere above, for use in medicine.

In a further aspect, the present invention provides a composition comprising a single immunoglobulin variable domain and a lipid-based carrier.

In the present invention, the said receptor, whose interaction with a ligand may be blocked, also may be referred to as the “ligand receptor”. This receptor being different from the T cell receptor.

The present invention advantageously provides new compositions and uses thereof which aim to provide alternative and/or improved formulations and delivery systems for antibodies and their fragments. According to embodiments of the present invention, an equivalent therapeutic effect may be achieved using a much lower amount of immunoglobulin or its fragment, compared to the prior art methods using free antibodies. Alternatively, compositions according to the present invention may show advantages in terms of increased shelf-life or stability, for instance with respect to temperature or low pH.

By “immunoglobulin or fragment thereof” it is meant an antibody or any polypeptide sequence derived from an antibody, particularly a fragment which is capable of specifically binding an antigen. Thus the term includes complete conventional antibodies, for example IgG, IgA or IgM, as well as chimeric and humanized forms of both complete antibodies and their fragments. More preferred are fragments such as Fab, Fab′, F(ab′)₂, Fv or single chain Fv (scFv fragments). Most preferred are fragments containing a single immunoglobulin variable domain, otherwise known as “domain antibodies”.

By “single immunoglobulin variable domain” or SVD it is meant a fragment that comprises a single V_{H}, V_{HH} or V_{L} region which is capable of specifically binding an antigen. Preferably the single immunoglobulin variable domain is a V_{H} domain, or a V_{HH} domain. A single immunoglobulin variable domain is typically a folded polypeptide domain which comprises sequences characteristic of immunoglobulin variable domains and which specifically binds an antigen (i.e. usefully with a dissociation constant of 500 nM or less). A “single immunoglobulin variable domain” therefore includes complete antibody variable domains as well as modified variable domains, for example in which one or more loops have been replaced by sequences which are not characteristic of antibody variable domains or antibody variable domains which have been truncated or comprise N- or C-terminal extensions, as well as folded fragments of variable domains which retain a dissociation constant of 500 nM or less (e.g., 450 nM or less, 400 nM or less, 350 nM or less, 300 nM or less, 250 nM or less, 200 nM or less, 150 nM or less, 100 nM or less) and the target antigen specificity of the full-length domain. A “domain antibody” or “dAb” is equivalent to a “single immunoglobulin variable domain polypeptide” as the term is used herein. Thus the immunoglobulin fragment may consist essentially of a single immunoglobulin variable domain, for example a single V_{H} or V_{HH} domain.

The phrase “single immunoglobulin variable domain polypeptide” encompasses not only an isolated single immunoglobulin variable domain, but also larger polypeptides that comprise one or more monomers of a single immunoglobulin variable domain polypeptide sequence. Such larger polypeptides comprising more than one monomer of a single immunoglobulin variable domain polypeptide are in noted contrast to scFv polypeptides which comprise a V_{H} and a V_{L} domain that cooperatively bind an antigen molecule. The monomers in the polypeptides described herein can bind antigen independently of each other. Thus where reference is made herein to compositions comprising a single immunoglobulin variable domain, the composition may in certain embodiments comprise single immunoglobulin variable domain polypeptides as defined above, i.e. a polypeptide comprising or consisting of more than one monomer of a single immunoglobulin variable domain.

The immunoglobulin or fragment thereof may be derived from any species, but is preferably a human immunoglobulin or fragment thereof, i.e. derived from a human germline immunoglobulin region. In further preferred embodiments, the immunoglobulin or fragment thereof is derived from a camelid, for example from a camel or llama, including chimeric or humanized forms derived from such species. Most preferred are human or llama single immunoglobulin variable domains, especially human V_{H} or camelid V_{HH} domains.

As used herein, the phrase “specifically binds” refers to the binding of an antigen by an immunoglobulin variable domain with a dissociation constant (Kd) of 1 μM or lower as measured by surface plasmon resonance analysis using, for example, a BIAcore surface plasmon resonance system and BIAcore kinetic evaluation software (e.g., version 2.1). The affinity or Kd for a specific binding interaction is preferably about 500 nM or lower, more preferably about 300 nM or lower.

As used herein, the term “antigen” refers to a molecule that is bound by an antibody or a binding region (e.g. a variable domain) of an antibody. An antigen can be a peptide, polypeptide, protein, nucleic acid, lipid, carbohydrate, or other molecule. Generally, an immunoglobulin variable domain is selected for target specificity against a particular antigen.

By “lipid-based carrier” it is meant any lipid-containing material, into which the immunoglobulin or fragment thereof can be stably incorporated for delivery or administration. Preferably the lipid-based carrier comprises at least 50%, at least 75%, at least 90%, or most preferably at least 95% by weight of a lipid, in the absence of the immunoglobulin or fragment thereof. Thus the lipid-based carrier may comprise any lipid-containing supramolecular assembly, including a micelle, a lamellar structure, a liposome or other lipid structure.

Preferably the lipid-based carrier is a microparticulate material, for example a vesicle-containing material, including unilamellar vesicles and multilammelar vesicles. By “vesicle” it is intended to refer to any predominantly spherical lipid-based particles, including particles containing a lipid bilayer (for example liposomes) or micelles. Immunoliposomes suitable for use in the present invention may in general be prepared as described in U.S. Pat. No. 6,214,388, especially columns 9-21, incorporated herein by reference.

Most preferably the lipid-based carrier comprises a plurality of micelles, otherwise known as immunomicelles in combination with the immunoglobulin or fragment thereof. Immunomicelles suitable for use in the present invention may be prepared as described in Torchilin et al., Proc. Nat. Acad. Sci. 100(10), 6039-6044 (2003), Torchilin et al., Biophys. Acta 1511, 397-411 (2001) or Torchilin et al., Proc. Nat. Acad. Sci. 98, 8786-8791 (2001), each incorporated herein by reference.

The lipid-based carrier may also comprise a mixture of two or more of the above-mentioned forms, in varying proportions, for example a mixture of micelles' and liposomes, or a mixture of micelles, liposomes and a lamellar structure. Preferably the lipid-based carrier comprises at least 50%, more preferably 75%, most preferably 90% by weight micelles. In alternative embodiments the lipid-based carrier comprises at least 50%, more preferably 75%, most preferably 90% by weight liposomes.

The lipid-based carrier may comprise any suitable lipid material. Preferably the lipid is an amphipathic lipid, for instance having hydrophobic and polar head moieties. More preferably, the lipid is a vesicle- or micelle-forming lipid, i.e. is capable of spontaneously forming vesicles such as liposomes, or most preferably micelles, in water. Lipid mixtures containing predominantly double-chain amphiphiles (i.e. lipids containing two long-chain acyl groups) tend to form liposomes whereas single-chain amphiphiles (such as lipids containing a single acyl chain) tend to form micelles. However both the number of acyl chains and the chain length influence the balance between micelle and liposome formation.

Micelles are spherical colloidal nanoparticles into which many amphiphilic molecules self-assemble. In water, hydrophobic segments of amphiphilic molecules form the core of a micelle, while hydrophilic parts of the molecules form the micelle corona. Preferably the micelles used in the present invention have a diameter of 1 to 200 nm, more preferably 5 to 50 nm.

The term “lipid-based carrier” is intended to encompass any amphiphilic lipid-like components, particularly those which may be used to form vesicles or micelles. Particularly preferred components of the carrier are phospholipids, for instance phosphatidylcholine (PC) or phosphatidylethanolamine (PE) or a mixture of PC and PE. In preferred embodiments phosphatidylcholine is used as a bulk constituent of the carrier, whereas PE is used as a component to which the immunoglobulin or fragment thereof is attached.

The lipid used in the lipid-based carrier may be modified to include a hydrophilic polymer such as polyethylene glycol (PEG). The polyethylene glycol moiety may be conjugated to the lipid. Addition of a hydrophilic polymer can increase the circulating lifetime of a liposome or micelle, leading to an increased half-life of the particle in the blood. Particularly preferred lipids are polyethylene glycol-derivatised lipids, in particular conjugates of PEG and diacyllipids, such as PEG-PE conjugates.

Preferably the immunoglobulin or fragment thereof is covalently linked to the lipid-based carrier, for instance by conjugation to a lipid, or PEG-lipid conjugates. More preferably the lipid-based carrier comprises a phospholipid-PEG-[immunoglobulin/immunoglobulin fragment] conjugate, for instance a PE-PEG-SVD conjugate, especially in the form of micelles.

The term “ligand” refers to a molecule capable of binding specifically to a particular “receptor” to form a bound complex. Thus the ligand and its corresponding receptor form a specific binding pair.

The compositions of the present invention may be used to block or inhibit the binding of a particular ligand to a particular receptor. In general, the uses and methods of the present invention may be performed in vitro, in vivo or ex vivo.

Preferably the compositions of the present invention are used to block ligand-receptor interactions which are involved in, or which mediate a human or animal disease. For instance, the immunoglobulin or fragment thereof may specifically bind to a ligand or receptor which is involved in disease induction or progression. The compositions of the present invention may thereby provide a therapeutic effect by blocking the ligand-receptor interaction to which the immunoglobulin or fragment thereof is reactive, rather than merely by targeting the composition to a cell type which expresses the ligand or receptor.

Preferably the ligand-receptor interaction (which is blocked by the present composition) normally mediates binding of a first cell to a second cell, a virus, a growth promoter, a cytokine or a hormone. For instance, a particular receptor may be expressed by the first cell, and is typically found on the cell surface (e.g. presented on the exterior of the cell membrane). A ligand which binds to that receptor may be expressed by a second cell (typically being located on the cell membrane), may be found on the surface of a virus (as a coat protein), or may be found in a free form (e.g. the ligand is found as the free molecule in an extracellular fluid, in the case of growth promoters, cytokines or hormones). The immunoglobulin or fragment thereof may bind specifically either to the ligand or to the receptor, provided that it interferes with the ligand-receptor interaction and thus signal transduction through the ligand-receptor complex. Preferably the immunoglobulin or fragment thereof binds to the ligand expressed by the second cell or virus, or directly to the growth factor, cytokine or hormone. Accordingly the present invention may be used to block the binding of one cell type to another cell type, or to block binding of a cell expressing a particular receptor to a free extracellular ligand.

In one preferred embodiment, the ligand-receptor interaction mediates binding of a first cell to an infective agent (in the absence of the composition of the present invention). Typically the first cell expresses a receptor to which the ligand, expressed by the infective agent, can bind. Preferably the immunoglobulin or fragment thereof binds to an antigen (ligand) expressed by the infective agent. The infective agent may be, for example, a virus, a bacterium, a protozoan or any other agent which is capable of infecting a eukaryotic, preferably a mammalian, more preferably a human cell.

In particularly preferred embodiments the infective agent is a rotavirus, human immunodeficiency virus (HIV), influenza virus, Helicobacter pylori or Candida albicans. In these embodiments, the immunoglobulin or fragment thereof preferably binds to an antigen selected from rotavirus VP4 adhesin, HIV gp20, influenza haemagglutinin, Helicobacter pylori BabA adhesin or Candida albicans SAP2 or MP65 virulence factor.

Amino acid sequences for these target antigens are known. For example, the sequences of some of the above-mentioned antigens are described in Kobayashi et al., Arch Virol. 1991; 121(1-4):153-62, Gorziglia et al., J. Virol., July 1992, 4407-4412, Vol 66, No. 7, De Bernadis F. et al. J. Infect. Dis. 1999, 179, 201-208, liver et al., Science. 1998 Jan. 16; 279(5349):373-7 and La Valle et al. Infect. Immun. 2000, 68, 6777-6784, each incorporated herein by reference. Given the amino acid sequence of the antigen, a skilled person can generate antigen for use in generating and selecting immunoglobulin polypeptides that specifically bind the antigen, using known techniques.

Methods for the generation and selection of antibodies or fragments thereof which bind to a particular antigen are known in the art and include in particular in vitro techniques for selecting binding antibodies or fragments from large libraries of polypeptides. One particularly preferred technique is phage display, which enables antibody fragments specific for a chosen target to be isolated without the need for animal immunization. Alternatively, an animal, preferably a camelid such as a llama, may be immunized with the antigen, leading to the selection of a hybridoma clone expressing a monoclonal antibody specific for the antigen, as is well known. Suitable techniques for generating, selecting and purifying immunoglobulins and their fragments, in particular single immunoglobulin variable domains, are discussed in Hudson et al. Nature Medicine 9(1), 129-133 (2003) and Holt et al. Trends in Biotechnology 21 (11), 484-490 (2003) and WO 2005/035572, especially pages 27-47, each of which are incorporated herein by reference.

As mentioned above, the present invention may be used to block ligand-receptor interactions in vitro, for instance in procedures involving cultured cells or tissues, or other experimental systems. However, the compositions of the present invention are preferably used in in vivo methods for the prevention or treatment of human or animal disease, especially infectious diseases, cancer, autoimmune diseases or inflammatory conditions. Examples of such conditions include gastric ulcer, chronic vaginal candidiasis, influenza, rotavirus and HIV infections, breast cancer, prostatic cancer, secondary tumour deposits dependent on angiogenic growth factors, thyrotoxicosis and asthma. The efficacy of compositions for use in the prevention or treatment of these conditions can be assessed using known in vitro tests or animal models.

Pharmaceutical compositions comprising the compositions of the present invention, typically in the form of immunomicelles or immunoliposomes, may be prepared according to standard techniques and further comprise a pharmaceutically acceptable excipient. Generally, normal saline will be employed as the pharmaceutically acceptable excipient. Other suitable excipients include, e.g., water, buffered water, 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the lipid-containing compositions of the present invention (typically liposome or micelle suspensions) may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

The concentration of the lipid-based carrier, typically immunoliposomes or immunomicelles, in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. Compositions comprising irritating lipids may be diluted to low concentrations to lessen inflammation at the site of administration.

The amount of the composition, typically an immunoliposome or immunomicelle, administered will depend upon the particular immunoglobulin or fragment thereof used, the disease state being treated, and the judgement of the clinician.

Generally the amount of the composition administered will be sufficient to deliver a therapeutically effective dose. The quantity of immunoliposomes necessary to deliver a therapeutically effective dose can be determined by uptake assays as are known in the art. As mentioned above, the use of a lipid-based carrier such as a liposome or micelle can typically reduce the amount of the immunoglobulin or fragment thereof which is necessary to produce a therapeutic effect, for instance to less than 50% less than 10% or less than 1% of the dose required in the absence of the lipid-based carrier. Typical immunoliposome or immunomicelle dosages will generally be between about 0.01 and about 50 mg per kilogram of body weight, preferably between about 0.1 and about 10 mg/kg of body weight per day.

The compositions of the present invention may be administered by any suitable route, for instance intravenous or parenteral. However, an advantage of the present compositions, especially those containing single immunoglobulin variable domains, is that because of their resistance to acid and proteolysis, they are suitable for oral administration.

Preferably, the pharmaceutical compositions are administered parenterally, i.e., intraarticularly, intravenously, intraperitoneally, subcutaneously, or intramuscularly. More preferably, the pharmaceutical compositions are administered intravenously or intraperitoneally by a bolus injection. Particular formulations which are suitable for this use are found in Remington's Pharmaceutical Sciences, Mack Publishing Company, Philadelphia, Pa., 17th ed. (1985).

The invention will now be described with reference to the following more specific embodiments.

Preferably the lipid-based carrier comprises a micelle.

A preferred embodiment of the present invention provides micelles bearing surface antibody (immunomicelles) which optimizes the targeting of cells bearing one or more characteristic surface markers, so as to block any interaction between that cell and another cell, microbe or a molecular structure such as a growth promoter or hormone. Although the surface antibody can be any antibody fragment such as scFv and Fab, the preferred formulations comprise a single variable domain (SVD) of an antibody heavy or light chain or any combination of SVDs, specific for the target cell characteristic marker(s). This antibody fragment is presented on the surface of a vesicle-forming lipid micelle.

The variable domains of the antibody heavy and light chains contain the structures which contact antigen. The isolated single variable domains (SVDS) can be engineered (Holt et al., Trends Biotechnol. 2003, 21, 484). They can bind to their cognate site on the antigen to block its interaction with another cell or molecule. SVDs have been shown to block the interaction of the specific sites on a number of microbes which bind to their cognate receptors on epithelial cells prior to colonization. Scaling up the amount of SVDs needed for treatment of humans requires particularly large quantities with cost and manufacturing disadvantages. Embodiments of the present invention overcome this problem by presenting the SVDs as a dense monolayer on the surface of lipid micelles, so that only a tiny fraction of that amount is needed.

The single variable domain antibody fragments are economic to produce in bulk and are very robust in their stability to environmental conditions. The SVD immunomicelles are extremely economic to use relative to the variable domain fragments in a soluble monomeric form because the micelle surface presents a monolayer consisting of a flexible array of fragments with extremely high avidity for the target due to their multivalent attachments.

The present invention provides novel single variable region domain antibody fragments (V_{H}) presented as a dense array on the surface of lipid micelles optimized for delivery to characteristic receptors on the target cell. They are capable of blocking the interaction of infectious agents with their cognate receptors on target cells or of blocking the interaction of a hormone or growth factor or cytokine with its receptor, or of sterically inhibiting the interaction of one body cell with another. SVDs derived from affinity matured human V_{H} phage libraries or derived from high affinity heavy chain antibodies from immunized llamas, are preferred species.

The SVDs utilized in the present invention have many advantages over intact antibodies, and even Fab or scFv fragments:

1) They can be expressed economically at high levels (e.g. g/L by fermentation in Pichia pastoris).
2) Relative to liposomes, micelles are more stable because there is not a complex bilayer lipid structure to maintain.
3) Relative to the efficacy of monomeric soluble antibodies, the dense array of antibodies on the micelle surface has an extremely high avidity resulting from the multiple bonds linking each micelle to its target cell. This is the so-called ‘bonus effect of multivalency’ (Roitt, Essential Immunology, 10th edn., 2001, p. 90). Note that this advantage results from the use of a lipid-based carrier, especially micelles as carriers, and is thus applicable to immunoglobulin fragments other than SVDs, although SVDs are particularly preferred. Furthermore, the lateral mobility of the immunoglobulins, especially SVDs in the lipid membrane provides the flexibility to align them geometrically in 2-dimensional space with their target molecules on the cell surface.
4) As a consequence of this much greater efficacy of the immunomicellular presentation of the antibody relative to the soluble monomer, very great economies for the provision of therapy are possible. Typically the amount of antibody required in the micellar form is of the order of not more than 1/100 of that needed if the soluble form is used.
5) Relative to the use of whole antibody molecules or Fab fragments, the SVD fragments are more readily expressed at high levels and lipid based carriers, especially immunomicelles containing them are robust and have a prolonged shelf-life because of their resistance to aggregation, proteolysis and denaturation so that the immunomicelles are essentially unaffected by temperature, therefore not requiring refrigeration. Their ability to withstand low pH makes them resistant to gastric breakdown thereby facilitating oral administration.
6) Furthermore, because of their small size, the dimensions of the immunomicelles will be smaller than those bearing whole immunoglobulin or Fab fragments with clear advantages for tumour access; the small size also enables more fragments to be packed onto the surface of each individual micelle. Another advantage of small size for tumour imaging is their rapid clearance from the body.
7) As each SVD fragment comprises a single variable domain, it is possible to create pairings of V_{H} and V_{L} fragments of different specificity, or immunomicelles bearing V_{H}s of two different specificities so providing dual targeting micelles for specific tumour therapy which can bind two completely different targets, even two different epitopes, not possible with conventional immunoglobulins and more difficult to achieve with recombinant antibody fragments, such as Fabs and scFvs.

The micelles of the present invention may be used to provide a therapeutic effect even in the absence of any additional pharmaceutical agent. However, in certain embodiments the hydrophobic core of the micelles of the present invention may additionally be employed as a cargo space to deliver one or more therapeutic agents such as poorly water-soluble drugs, such as taxol. Micelle encapsulation increases bioavailability of poorly soluble drugs, protects them from destruction in biological surroundings, and beneficially modifies their pharmacokinetics and biodistribution. Thus the invention also provides for internalization into target cells bearing the characteristic surface markers, of micelles including polyethylene glycol derivatized lipid and containing lipophilic or amphiphilic materials, e.g. poorly soluble drugs, such as taxol.

In alternative embodiments, the invention provides for the use of small liposomes, particularly for entrapment of hydrophilic materials including pharmaceutical agents such as siRNA for delivery to the cell interior to block specific events. Several publications have described the use of immunoliposomes to optimize internalization into target, cells (e.g. AU2003241028, US2001016196, WO0050008, EP1352662, WO9738731, U.S. Pat. No. 6,214,388, WO9614864, U.S. Pat. No. 5,786,214, IN182550, WO9103258, U.S. Pat. No. 4,957,735). U.S. Pat. No. 6,214,388 [1996] describes immunoliposomes bearing the Fab′ fragment of monoclonal antibodies targeting characteristic cell surface markers. Liposomes with intact monoclonal antibodies (Boot E. P. et al. Arthritis Res. Ther., 2005, 7(3), R604-15) can target CD134 for specific drug delivery to activated T-helper cells.

In one preferred embodiment, this invention provides for SVD-immunomicelles that optimize the blocking of the attachment of C. albicans to vaginal epithelium through its virulence factor SAP2 (De Bernardis et al. J. Infect. Dis. 1999, 179, 201) to treat chronic vaginal candidiasis. The immunomicelles comprise the affinity matured V_{H} of a human anti-candida SAP2 with a C-terminal hydrophobic tail of alanine or a palmitoyl group, or a C-terminal cysteine for coupling covalently. Particularly preferred as targets are the BabA adhesin on H. pylori, the organism linked to gastric ulcers and cancer, and VP4 adhesin on rotavirus responsible for widespread diarrhea in the young. Also preferred as targets are growth factor receptors on cancer cells such as Her2 on breast cancer, receptors for angiogenic factors on tumour vascular endothelium, and cytokine receptors on cells such as macrophages in synovial inflammatory sites in patients with rheumatoid arthritis. Other targets are virulence factors on viruses such as rotavirus VP4, influenza haemagglutinin and HIV gp120.

This invention also provides for a method as defined above, comprising a further step of internalization of the lipid-based carrier, for instance an immunomicelle, into a cell bearing a characteristic cell surface marker, i.e. a cell expressing the ligand or receptor to which the immunoglobulin or fragment thereof is directed. The method preferably comprises contacting the cell with a targeting immunomicelle bearing a lipid derivative of polyethylene glycol. The micelle may include any suitable therapeutic agent, preferably a poorly soluble drug, including but not limited to lipophilic and amphiphilic radioisotopes, anti-cancer drugs such as daunomycin, idarubicin, mitoxantrone, mitomycin, cisplatin and other Platinum II analogues, vincristine, epirubicin, aclacinomycin, methotrexate, etoposide, doxorubicin, cytosine arabinoside and fluorouracil, polypeptides and antibiotics.

The invention can be extended to micelles formed from single domain fragments derived from the variable regions of the alpha/beta chains comprising the T-lymphocyte antigen receptors which recognize cells expressing a combination of their major histocompatibity complex (MHC) molecule with an internally derived processed protein or other fragment (Roitt's Essential Immunology, 11^{th} Edition, 2006, p. 63). In particular, micelles bearing these so-called TCR nanobodies could deliver appropriate therapeutic drugs such as antibiotics to internally infected macrophages. Others could deliver cytotoxic drugs to cancer cells or virally infected cells, or dendritic cells or B-cells crucially presenting antigen in autoimmune diseases thus substituting for cytotoxic T-cells. This would be specially advantageous in viral respiratory diseases where inhalation of the micelles would provide direct contact with infected cells and also in circumstances where regulatory T-cells are damping down the action of the cytotoxic T-cells Micelles bearing gamma/delta TCR nanobodies could have special application for combating mucosal infections. Attempts to exploit the potential therapeutic activity of monoclonal soluble T-cell receptors suffer from the very low innate affinity for the MHC antigen complex, a disadvantage which is overcome by the multivalence of the micelles predicated in the current invention. Micelles containing antibody molecules covering the surface may be constructed by anyone skilled in the art using standard methods. For example, lipid chains may be conjugated to certain amino acid residues on the peptide chain of a SVD (including, but not limited, to primary amino, carboxyl, hydroxy or sulphydryl groups) where said lipid chains may be derived from long-chain hydrocarbon fatty acids, (either straight-chain or branched, saturated or unsaturated, unsubstituted or substituted with halogen atoms). The antibody-lipid conjugates thus formed may be incorporated into micelles either by incubating with pre-formed micelles, or by incubating in a detergent solution containing solubilised amphiphilic components which act as precursors of micelles, followed by dialysis to remove the detergent.

A second method of constructing immunomicelles is to conjugate, using standard methods, antibody molecules directly to pre-formed micelles (e.g. Torchilin V. P. et al. Proc. Natl. Acad. Sci. 2003, 100, 6039) which contain amphiphiles presenting functional groups on their surface capable of linking to residues on the protein chain, including, but not limited to, primary amino, carboxyl, hydroxy or sulphydryl groups.

The micelles may range in size from 10 to 200 nm, and the coverage of the antibody on the micelle may vary from 1 to 10% of the total surface area.

Amphiphile components which may be used to form micelles are known in the art, and can include pegylated lipid ethers, pegylated lipid esters, bile salts, fatty acids and their salts, sodium docusate, palmitoyl choline, palmitoyl carnitine, long-chain hydrocarbons containing positive or negative charge when ionized, phospholipids, lysopholipids, pegylated phospholipids, triglycerides, lipid-conjugated oligopeptide, substituted derivatives, homologues, analogues and mixtures thereof.

It will be appreciated by those skilled in the art that an immunoglobulin or fragment thereof as referred to in the embodiments of the invention described anywhere herein may be substituted by a T cell receptor or fragment thereof, in accordance with the invention.

### EXAMPLES

### Example 1

Immunomicelles are prepared by a procedure using polyethylene glycol-2000-phosphatidylethanolamine (PEG-2000-PE) with the free PEG terminus activated by p-nitrophenylcarbonyl (pNP). Micelles are prepared from PEG-PE with the addition of a small fraction of pNP-PEG-PE. The PE residues form the micelle core, whereas the pNP groups allow for attachment of aminogroup-containing ligands via the formation of a urethane (carbamate) bond.

PE and PEG-2000-PE are commercially available from AVanti Polar Lipids. pNP-PEG-PE is synthesized as described in Torchilin et al., Biophys. Acta 1511, 397-411 (2001). A lipid film is prepared by removing chloroform from a mixed solution of pNP-PEG-PE under vacuum. To form micelles, the film is rehydrated at 50° C. in a 5 mM Na citrate-buffered saline, pH 5.0, and vortexed for 5 min.

A llama V_{HH} anti-rotavirus VP4 is obtained by screening a library of V_{HH} domains derived from immunized llamas, using the VP4 antigen as described in Kobayashi et al., Arch Virol. 1991; 121(1-4):153-62 or Gorziglia et al., J. Virol., July 1992, 4407-4412, Vol 66, No. 7. The llama V_{HH} anti-rotavirus VP4 is incorporated into pNP-PEG-PE micelles of average diameter 30 nm using the method described in Torchilin V P et al., PNAS (2003); 100; 6039-6044. A culture of the MA104 mammalian cell line infected with 10⁶ CK5 rotavirus particles is incubated with this micellar construct to compare its efficacy in inhibiting viral plaque formation with the soluble form of the SVD.

### Example 2

Adherence of Candida albicans at a concentration of 1.5×10³ cells/ml in M199 liquid medium, to polystyrene plastic is measured by counting colonies (San Millan R. et al. Microbiology 1996, 142, 2271). An anti-SAP2 SVD is obtained by phage display screening of a human immunoglobulin library, panning against the SAP2 antigen, as described in De Bernadis F. et al. J. Infect. Dis. 1999, 179, 201-208. The soluble form of the single domain (SVD) human anti-candida SAP2 clone is compared with a micellar suspension containing the same SVD to compare relative inhibitory activity.

### Example 3

A single domain human antibody V_{H} fragment specific for Helicobacter pylori BabA is obtained by phage display screening of a human immunoglobulin library, panning against the BabA adhesin as described in liver et al., Science. 1998 Jan. 16; 279(5349):373-7. The binding of Helicobacter pylori to immobilized Lewis b conjugated to human serum albumin through its BabA adhesin is measured by surface plasmon resonance (Hirmo S. et al. Analyt. Biochem. 1998, 257, 63). The soluble form of a cloned single domain human antibody V_{H} fragment specific for BabA is compared with the micellar form to evaluate their relative inhibitory properties.

### Example 4

An anti-SAP2 SVD is obtained as described in Example 2. Oophorectomized rats maintained under pseudoestrus and inoculated with 10⁷ yeast cells in 0.1 ml saline (Cassone et al. Curr. Mol. Med, 2005, 5, 377) are injected intravaginally with the soluble and micellar forms of the anti-SAP2 SVD. The two SVD preparations are compared for their relative ability to accelerate clearance of the Candida organisms.

### Example 5

Four-day old mouse pups are fed 10 μl of either a soluble or micellar form of an SVD anti-rotavirus VP4, obtained as described in Example 1, twice daily during 5 days using a pipette tip. After the first day, pups are infected with 10 μl of rotavirus suspension containing 2×10⁷ pfu and evaluated for clinical diarrhea by daily palpation of the abdomen during the 6 days of the experiment. The relative efficacy of the soluble and micellar forms of the SVD are thereby compared

### Example 6

Soluble and micellar forms of an SVD anti-HIV gp120 obtained as described in Example 1 are compared for their ability to block the infection of a CD4-bearing cell line with the original strain of HIV providing the gp120 for selection of the SVD and with another strain only weakly neutralized by the SVD monomer.

### Example 7

The N-terminal variable region domains of the alpha and beta chains of the T-cell receptor of a murine T-cell line cytotoxic for an influenza infected cell target are cloned using standard procedures. The clones are engineered to contain a C-terminal cysteine and if necessary, to stabilize the T-cell receptor heterodimer, the fos and jun gene sequences which give rise to the leucine zipper segments, will be incorporated. Alternatively, the variable region domains can be joined by an appropriate linker sequence. The clones are expressed in E. coli, and the monomers constructed into micelles as described in Example 1 but in this case, additionally incorporating an anti-viral drug such as poly-IC which would have the dual effect of initiating interferon synthesis and increasing MHC surface expression. To ascertain whether this micellar construct can substitute for a cytotoxic T-cell, the cytotoxic efficacy of the micelles against the target cells is compared with that of the original cytotoxic cell line providing the T-cell receptor genes and a cytotoxic cell line with an irrelevant specificity acting as a negative control.

1. A composition comprising an antigen recognizing molecule or fragment thereof and a lipid-based carrier, wherein said composition is capable of blocking an interaction between a ligand and a receptor.

2. The composition according to claim 1 wherein the antigen recognizing molecule is a T cell receptor or fragment thereof.

3. The composition according to claim 1 wherein the antigen recognizing molecule is an immunoglobulin or fragment thereof.

4-6. (canceled)

7. The composition of claim 1, wherein the lipid-based carrier comprises a micelle.

8. The composition of claim 1, wherein the lipid-based carrier comprises a polyethylene glycol-derivatised lipid.

9. The composition of claim 1, wherein the antigen recognizing molecule or fragment thereof is covalently linked to the lipid-based carrier.

10. The composition of claim 1, wherein the ligand-receptor interaction mediates binding of a first cell to a second cell, a virus, a growth promoter, a cytokine or hormone.

11. The composition of claim 1, wherein the ligand-receptor interaction mediates binding of a first cell to an infective agent.

12. The composition of claim 11, wherein the infective agent is a rotavirus, HIV, influenza virus, Helicobacter pylori or Candida albicans.

13. The composition of claim 12, wherein the antigen recognizing molecule or fragment thereof binds to an antigen selected from rotavirus VP4 adhesin, HIV gp20, influenza haemagglutinin, Helicobacter pylori BabA adhesin or Candida albicans SAP2 or MP65 virulence factor.

14. A method of preventing or treating a disease, comprising administering to a subject a therapeutically effective amount of a composition comprising an antigen recognizing molecule or fragment thereof and a lipid-based carrier, wherein the disease is mediated by binding of a ligand to a receptor, and the composition is capable of blocking binding of the ligand to the receptor.

15. The method of claim 14 wherein the antigen recognizing molecule is a T cell receptor or fragment thereof.

16. The method of claim 14 wherein the antigen recognizing molecule is an immunoglobulin or fragment thereof.

17. The method of claim 14, wherein the disease is an infectious disease, cancer, an autoimmune disease or an inflammatory condition.

18. The method of claim 14, wherein the antigen recognizing molecule or fragment thereof binds to the ligand or the receptor.

19. The method of claim 14, wherein the composition is administered orally.

20. A method for inhibiting an interaction between a ligand and a receptor, comprising contacting the ligand and/or receptor with a composition comprising an antigen recognizing molecule or fragment thereof and a lipid-based carrier, wherein the antigen recognizing molecule or fragment thereof binds to the ligand and/or receptor, thereby inhibiting the interaction between the ligand and the receptor.

21. A method for inhibiting an interaction between a ligand and a receptor according to claim 20 wherein the antigen recognizing molecule is a T cell receptor or fragment thereof.

22. A method for inhibiting an interaction between a ligand and a receptor according to claim 20 wherein the antigen recognizing molecule is an immunoglobulin or fragment thereof.

23-25. (canceled)

26. A composition comprising an immunoglobulin or fragment thereof and a lipid-based carrier, wherein the composition is capable of inhibiting binding of a ligand to a receptor, binding of the ligand to the receptor being associated with induction or progression of a human or animal disease.

27. A composition as defined in claim 26, for use in medicine.

28. A composition comprising a single immunoglobulin variable domain and a lipid-based carrier.

29. A composition according to claim 26, wherein the immunoglobulin fragment or the single immunoglobulin variable domain comprises a V_{H} or V_{HH} domain.

30. A composition according to claim 26, wherein the immunoglobulin, the fragment thereof or the single immunoglobulin variable domain is derived from human or llama.

31. A composition according to claim 26, wherein the lipid-based carrier comprises a micelle.

32. A composition according to claim 26, wherein the lipid-based carrier comprises a polyethylene glycol-derivatised lipid.

33. A composition according to claim 26, wherein the immunoglobulin, the fragment thereof or the single immunoglobulin variable domain is covalently linked to the lipid-based carrier.

34. A composition according to claim 26, wherein the immunoglobulin, the fragment thereof or the single immunoglobulin variable domain binds to a hormone, a growth promoter, a cytokine, or an antigen associated with an infective agent.

35. A composition according to claim 26, wherein the composition does not comprise a further therapeutic agent carried by the lipid-based carrier.

36. A composition according to claim 26, wherein the composition comprises a further therapeutic agent carried by the lipid-based carrier.

37. A composition according to claim 30, wherein the lipid based carrier comprises a micelle and the further therapeutic agent is a poorly water-soluble drug carried by the core of the micelle.

38. A composition according to claim 28, comprising two or more single immunoglobulin variable domain polypeptides, each polypeptide being capable of binding specifically to a different antigen.

39. A composition according to claim 26, wherein the lipid-based carrier comprises a liposome.

40. A composition according to claim 26, wherein the immunoglobulin fragment or the single immunoglobulin variable domain comprises a V_{L} domain.

41. A composition comprising a T cell receptor or fragment thereof and a lipid based carrier.

42. (canceled)
